(19) United States
(12) Patent Application Publication
(10) Pub. No.: US 2024/0238023 A1
(43) Pub. Date: Jul. 18, 2024

(54) ARTHROSCOPIC BONE WAX TOOL

(71) Applicant: MDS, LLC, Boulder, CO (US)

(72) Inventors: Omer Mei-Dan, Boulder, CO (US); Jacob Segil, Boulder, CO (US); Louis Piccione, Broomfield, CO (US); Sydney Levy, Berthoud, CO (US)

(21) Appl. No.: 18/415,466

(22) Filed: Jan. 17, 2024

Related U.S. Application Data
(60) Provisional application No. 63/439,820, filed on Jan. 18, 2023.

(51) Int. Cl. A61B 17/88

(52) U.S. Cl. CPC A61B 17/8819; A61B 17/8811; A61B 17/8822

(57) ABSTRACT

Embodiments are directed to a bone wax delivery tool for arthroscopic procedures. The bone wax delivery tool can include a cannula defining an inner cavity configured to hold a bone wax material and a plunger that extends along the inner cavity of the cannula. The plunger can include an end portion configured to contact the bone wax material and be configured to move between a first position and a second position within the cannula. The bone wax delivery tool can include an actuation mechanism coupled to the plunger. The actuation mechanism can cause the plunger to move from the first position to the second position and expel an amount of the bone wax material from the cannula in response to actuation and cause the plunger to move from the second position to the first position in response to release of the actuation mechanism.

### CROSS-REFERENCE TO RELATED APPLICATION

This application is a nonprovisional of, and claims the benefit under 35 U.S.C. § 119(e) of, U.S. Provisional Patent Application No. 63/439,820, filed Jan. 18, 2023, titled “Arthroscopic Bone Wax Tool” the contents of which are incorporated herein by reference.

### FIELD

The described embodiments relate generally to surgical tools for arthroscopic procedures and more particularly, the present embodiments relate to arthroscopic tools for delivering bone wax material.

### BACKGROUND

Arthroscopic procedures use a camera that is inserted through an incision to view anatomical structures inside a patient. Surgical tools inserted through additional incision sites and are used to perform various treatments on a patient. Arthroscopic procedures can include using tools such, as a burr, to shape or otherwise modify bone and other tissue at a surgical site. Using a tool to remove bone or other tissue can cause the bone to bleed and cause the arthroscopic cavity to fill with blood, which may be undesirable.

### SUMMARY

Embodiments are directed to a bone wax delivery tool for arthroscopic procedures that includes a cannula defining an inner cavity configured to hold a bone wax material and a plunger that extends along the inner cavity of the cannula. The plunger can include an end portion configured to contact the bone wax material and configured to move between a first position and a second position within the cannula. The bone wax delivery tool can include an actuation mechanism coupled to the plunger and configured to cause the plunger to move from the first position to the second position and expel an amount of the bone wax material from the cannula in response to actuation. The actuation mechanism can cause the plunger to move from the second position to the first position in response to release of the actuation mechanism.

Embodiments are directed to a bone wax delivery tool for arthroscopic procedures that can include a cannula defining an inner cavity configured to hold a bone wax material, a plunger positioned in the inner cavity of the cannula and an actuation mechanism coupled to the plunger. The actuation mechanism can be configured to cause the plunger to advance along the cannula and expel a first portion of the bone wax material from the cannula in response to a first actuation. The actuation mechanism can cause the plunger to advance along the cannula and expel a second portion of bone wax material from the cannula in response to a second actuation.

Embodiments include a bone wax delivery tool for arthroscopic procedures that includes a cannula defining an inner cavity configured to hold a bone wax material and a plunger that extends along the inner cavity of the cannula and configured to contact the bone wax material. The bone wax delivery tool can include a handle attached to the cannula and an actuation mechanism coupled to the plunger. The actuation mechanism can include an input structure positioned outside the handle and configured to move between a first position and a second position. The input structure can move from the first position to the second position and causes the plunger to expel a defined amount of the bone wax material from the cannula in response a press operation to the input structure and return to the first position in response to release of the input structure.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows an example of a bone wax delivery tool being used during an arthroscopic procedure;

FIGS. 2A and 2B show an example bone wax delivery tool;

FIG. 3 shows an exploded view of an example bone wax delivery tool as described herein;

FIG. 4 shows an example bone wax delivery tool;

FIG. 5 shows a detailed view of an end portion of the bone wax delivery tool shown in FIG. 4;

FIG. 6 shows an example bone wax delivery tool;

FIG. 7A shows an example bone wax delivery tool that can deliver multiple doses of a bone wax material;

FIG. 7B shows a detailed view of an end portion of the bone wax delivery tool shown in FIG. 7A;

FIG. 8 shows an example bone wax delivery tool that can deliver multiple doses of a bone wax material; and

FIGS. 9A and 9B shows an example bone wax delivery tool that includes a cutting mechanism.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

Embodiments disclosed herein are directed to surgical tools for arthroscopically delivering bone wax material at a surgical site. The surgical tools, which may also be referred to as bone wax delivery tools, can include a cannula that is configured to hold a bone wax material. The surgical tool can include a plunger and actuation mechanism that function to expel bone wax material from the canula and onto a bleeding portion of a bone.

Various orthopedic arthroscopic procedures can include cutting, repairing or otherwise modifying bone or surrounding tissue such as cartilage, bone marrow and so on, which can cause the bone to bleed. For example, osteoplasty can be performed on a femoral head to reshape the bone, which may be performed to treat an impingement or other condition. The osteoplasty can be performed arthroscopically and a bur may be used to cut away portions of the bone and reshape the femoral head. As portions of the bone are cut, blood passages may be exposed causing the bone to bleed. The bleeding can obstruct the surgeons view and/or may cause other undesirable effects. The bleeding may occur from multiple different passages within the bone. Traditional blood sealing techniques such as cauterizing cannot be used on the bone.

It can be desirable to precisely control the amount and/or location of bone wax applied to a bone, for example so that excess bone wax doesn't detach and float around in the surgical site. In some cases, the bone wax delivery tools described herein can be configured to expel a defined amount of bone wax (also referred to as a “dose”) onto the bone to block one or more blood passages. A bone wax delivery tool can have a cannula which is inserted into the patient, a plunger positioned at least partially within the cannula, bone wax material positioned in the cannula, and an actuation mechanism. The actuation mechanism can have an input structure that is actuated (e.g., pressed) by a user and causes the plunger to expel bone wax from the cannula. The actuation structure may be configured to cause the plunger to expel a defined amount of bone wax with each complete actuation (e.g., press and/or release operation). In some cases, the defined amount of bone wax material can be configured to roughly match a size of one or more blood channels within the bone. The surgeon may place the end of the tool near or contacting the blood channel and actuate the bone wax delivery tool to expel bone wax material over and/or into one or more blood passages. The bone wax material may prevent bleeding from the bone.

The term cannula can include a tube structure or other suitable structures that are introduced to a surgical site using arthroscopic techniques. For example, a cannula can include an elongated tube that is inserted into a patient through an incision in the patient's skin. The cannula can be configured to be inserted directly through an incision site and/or through a structure positioned in an incision such as an arthroscopic portal. As used herein, bone wax material can include any medical substance that is used to control bleeding from bone or surrounding tissue during a surgical procedure.

In some cases, the bone wax delivery tool can be configured to expel a single dose of material. For example, the bone wax delivery tool includes approximately one dose of bone wax and upon expelling that dose, the tool would need to be removed from the site and recharged with new bone wax or replaced with another bone wax delivery tool. In other cases, the bone wax delivery tool can include multiple doses of bone wax material. In these cases, the bone wax delivery tool may remain at the surgical site and multiple doses can be delivered to one or more locations on a bone.

The actuation mechanism can be configured to control the amount of bone wax material that is expelled from the device in a variety of ways. In some cases, the actuation mechanism can define a travel path of a user engagement structure such as a button or any other suitable structure. Each complete press of the button can be configured to expel a defined dose of bone wax material. In some cases, the actuation mechanism may allow the dose amount to be adjusted by the user, which can allow the user to increase or decrease the amount of bone wax material for each actuation cycle. Additionally or alternatively, the actuation mechanism can dispense an amount of bone wax based on an extent the user actuates the user input structure. For example, the amount of bone wax material that is expelled can be proportional to the amount that the user input structure is moved. Accordingly, in these cases, the surgeon or other medical personnel can directly control the amount of bone wax material expelled from the tool.

Bone wax material can have a waxy structure which may make it difficult to release from the bone wax tool. For example, the bone wax material may stick to itself making it difficult to separate a dose of material from a larger portion of the material in the bone wax tool. Additionally or alternatively, the bone wax material may stick to the tool which may make cause expelled bone wax to pull away from the bone or otherwise not securely adhere to the bone surface.

An end portion of the cannula can be configured to help release a dose of bone wax material from the bone wax delivery tool. In some cases, the tip of the cannula may be angled which may help a user to manipulate the tool after expelling a dose of bone wax material. For example, the angled tip may help facilitate a wiping or scraping motion to adhere the bone wax material to the bone. In other cases, the plunger and/or an internal surface of the cannula can be configured to help release the bone wax material. For example, the internal surface at the end of the cannula may define a ramped structure (e.g., angled internal surface between the sidewall and opening) and the end of the plunger may include a complimentary angled surface which contacts the ramped structure on the canula. Accordingly, when the cannula expels the bone wax material the angled surface on the plunger contacts the angled inner surface of the cannula helping to separate a dose of bone wax by having a lower amount of surface area in contact with the bone wax material. In other cases, the bone wax delivery tool may at least partially surround the bone wax material and advancing the plunger in the cannula (e.g., using the actuation mechanism) may cause the plunger to advance and/or cut a dose of the bone wax material to help expel the dose of bone wax material from the device.

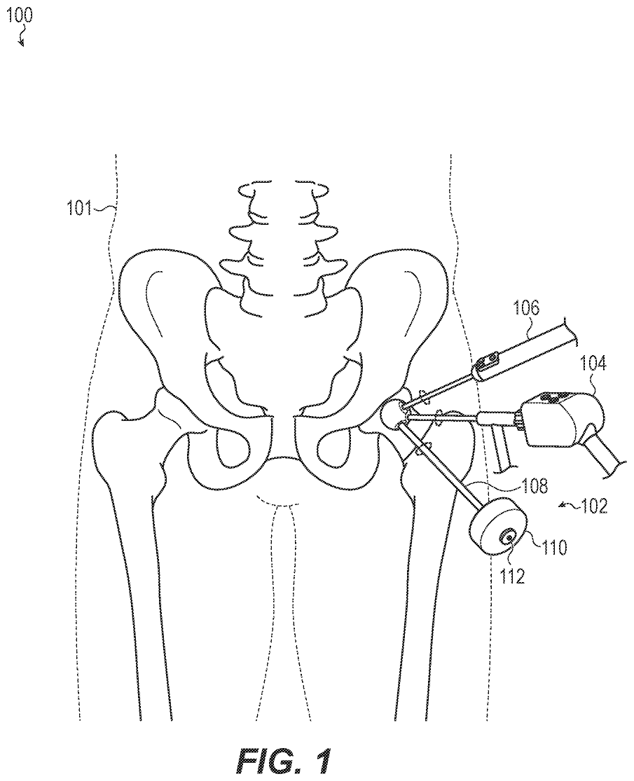

These and other embodiments are discussed below with reference to FIGS. 1-9. However, those skilled in the art will readily appreciate that the detailed description given herein with respect to these Figures is for explanatory purposes only and should not be construed as limiting.

FIG. 1 shows an example of a bone wax delivery tool 102 being used during an arthroscopic procedure 100 that is being performed on a patient 101. The arthroscopic procedure 100 can include the use of an arthroscopic camera 104, one or more surgical instruments 106 (one of which is shown for clarity), and the bone wax delivery tool 102. The arthroscopic camera 104 can be inserted through a first entry site and used to view the surgical site inside the patient 101. The instrument 106 can be inserted into the patient using a second entry site, but the instrument 106 can be inserted and removed from any of the entry sites. The one or more instruments 106 can include any instruments that are used in arthroscopic surgery or other surgical procedures such as a burr or other bone shaping tools, graspers, retrievers, biters, hooks, mallets, punches, and so on. The illustrated arthroscopic procedure 100 shows an example of a hip procedure, however, the devices and concepts described herein can be applied to other arthroscopic procedures such as shoulder, knee, and ankle procedures as well as other minimally invasive surgeries or other surgical operations where suture management would be beneficial.

The bone wax delivery tool 102 can be used to arthroscopically deliver bone wax material to the surgical site. The bone wax delivery tool 102 can include a cannula 108, which is inserted into the patient 101 through an incision site. The bone wax delivery tool 102 can include a handle 110 that is located outside the patient and an actuation structure 112 that causes bone wax material to be expelled from the cannula 108 and onto the bone.

The bone wax delivery tool 102 can include one or more doses of bone wax material as described herein. In some cases, the bone wax material can be located inside the cannula 108 and expelled from an end of the cannula 108 by an actuation mechanism. The actuation mechanism can include the actuation structure 112, which can be any suitable structure that can be actuated by the user. For example, the actuation structure 112 may include an end of a plunger that is pressed by the user such as a surgeon. Accordingly, the actuation mechanism can allow the user to expel the bone wax material onto a bone from a portion of the bone wax tool 102 that is located outside the patient.

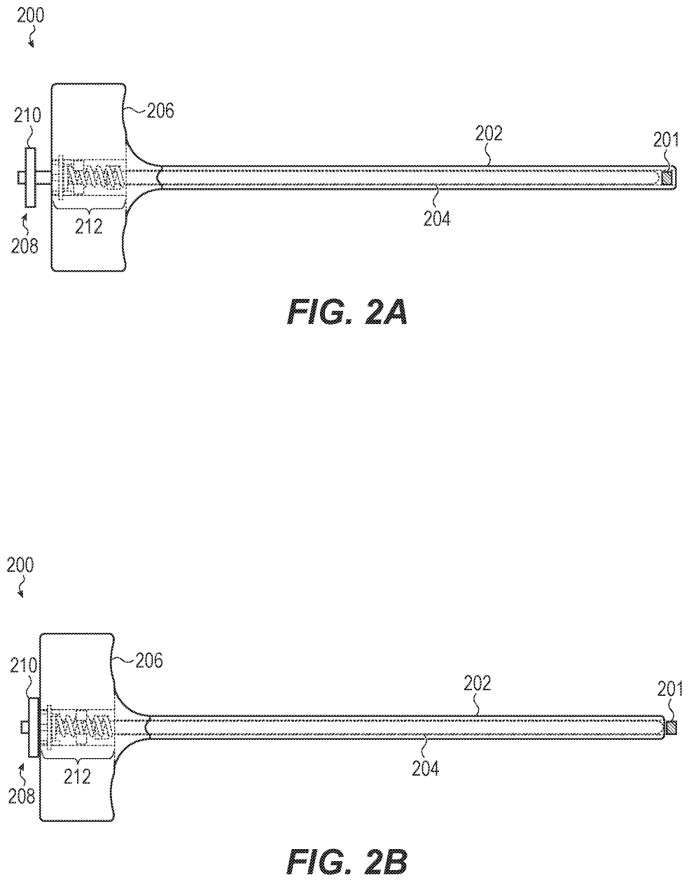

FIGS. 2A and 2B show an example bone wax delivery tool 200, which may be an example of the bone wax delivery tools described herein. FIG. 2A shows an example of the bone wax delivery tool 200 in an unactuated state and FIG. 2B shows an example of the bone wax delivery tool 200 in an actuated state. The bone wax delivery tool 200 can include a cannula 202, a plunger 204, a handle 206 and an actuation mechanism 208.

The cannula 202 can be coupled to the handle 206 and define an inner cavity. The plunger 204 can be positioned in the cavity. In some cases, the handle 206 may also define a cavity and the plunger can extend into and/or through the handle 206. In the un-actuated state, as shown in FIG. 2A a bone wax material 201 can be positioned in the inner cavity of the cannula 202 at a distal portion of the cannula 202. In some cases, the bone wax material 201 can include a single dose of material and substantially all of the bone wax material can be expelled during actuation of the actuation mechanism 208. In other cases, the bone wax material 201 can include a multiple doses of material that are contained within the cannula 202 as single piece of material, and actuation of the delivery tool 200 can cause a portion of the bone wax material 201 to be expelled from the cannula 202. In other cases, the multiple doses of bone wax material 201 can be divided into multiple discrete pieces of material, which may help separate a doses of the bone wax material 201 as it is expelled from the cannula.

The actuation mechanism 208 can include an input structure 210 for receiving a user input to the bone wax delivery tool 200 and an actuation control assembly 212 that controls motion of the plunger 204. In some cases, the input structure 210 can be part of the plunger 204 and/or coupled to or formed on an end of the plunger 204. The input structure 210 can be configured to be engaged by a user in particular way. For example, the input structure 210 can define a surface that is pressed by a user's thumb or finger. Although this is one example, and the input structure 210 can be configured in any suitable way. When a user actuates (e.g., presses) the input structure 210, the input structure can cause the plunger 204 to move along the cannula 202 to expel the bone wax material 201.

In some examples, the actuation mechanism 208 can include gearing, or other mechanical system to couple motion of the input structure 210 to the plunger 204. For example, the amount of bone wax material 201 that is needed to stop/reduce bleeding from a vessel may require very little motion when the plunger is 204 is directly attached to the input structure 210. Accordingly, the actuation mechanism 208 can include gearing, linkages or other structures that allow for greater motion of the input structure 210 to expel the same amount of the bone wax material 201. This may allow a user to have greater control over the delivery of the bone wax material 201 from the cannula 202.

In some cases, the actuation control assembly 212 can control the range of movement and movement path of the plunger 204 and/or the input mechanism 208. Additionally or alternatively, the actuation control assembly 212 can define a movement feel of the input structure 210, for example resistance of the actuation, clicking or other types of haptic feedback that are provided to a user. In some cases. the actuation control assembly 212 can limit the travel of the plunger 204, for example, so that an end of the plunger does not leave the cannula or has a defined positioned with respect to the end of the cannula 202, at the end of the actuation stroke.

As shown in FIG. 2A, the actuation control assembly 212 can bias the input structure 210 and plunger 204 in a first position, which may correspond to a retracted position. In the first state the bone wax material 201 may be positioned within the cannula 202. FIG. 2B shows the input structure 210 and plunger 204 in a second position. In the second position, the plunger 204 may move along the cannula 202 to expel bone wax material 201 from the end of the cannula 202. In some cases, the actuation mechanism may include a biasing element, such as a spring, that biases the input structure 210 and/or plunger 204 to the first position. Accordingly, upon a user releasing the input structure 210 the input structure 210 and plunger 204 may automatically move back to the first position.

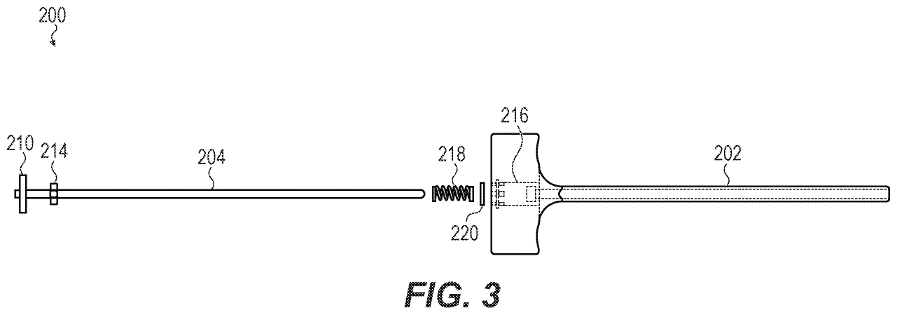

FIG. 3 shows an exploded view of an example bone wax delivery tool 200 as described herein. The actuation mechanism 208 (shown in FIGS. 2A and 2B) can include a key 214 the is coupled to the plunger 204, a recess 216 formed in the handle 206, a biasing element 218 and a stop 220.

The actuation mechanism 208 can be configured to control movement of the plunger 204 and/or input structure 210 during actuation and release operations. In some cases, the key 214 can be configured to prevent rotation movement of the plunger 204 with respect to the cannula 202. For example, the key 214 may include a non-circular profile that fits into the recess 216. The recess 216 may include a complimentary shape that allows the key 214 to move axially within the handle 206 while preventing rotation of the key 214 and the plunger 204.

In other cases, the key 214 and the recess 216 may be configured to cause the plunger 204 to rotate during actuation (movement of the plunger 204 from the first position to the second position) and/or release (movement of the plunger 204 from the second position to the first position) of the input structure 210. For example, the key 214 can have a first outer profile and the recess 216 can have a second profile that acts as a guide for the key 214. In some cases, the second profile of the recess 216 may twist along the axial direction of the plunger 204 thereby causing the plunger 204 to rotate when actuated and/or released.

In some cases, the recess 216 may include a first track/profile that the key 214 runs along when actuated and a second track/profile that the key 214 runs along when released. This can cause the plunger 204 to have different movement during actuation and release. For example, actuation of the input structure 210 may cause the key 214 to run along a first track in the recess 216 that causes the plunger 204 to move axially along the cannula 202 withing rotating. Release of the input structure 210 may cause the key 214 to run along a second track in the recess 216 that causes the plunger 204 to rotate as it retracts to the first position. In some cases, the rotation of the plunger during retraction may help release the bone wax material from the bone wax delivery tool 200. Further the rotation movement of the plunger 204 may cause a wiping/spreading motion of the bone wax material against the bone (e.g., on and/or within a blood passage) which may help adhere the bone wax material to the bone to stop or reduce bleeding.

The biasing element 218 can be configured to cause the plunger 204 and/or input structure 210 to the first position (e.g., unactuated position). In some cases, the biasing element 218 can include a spring positioned in the handle 206 and/or cannula 202 the compresses between the key 214 and a bottom surface defined by the handle 206 and/or cannula 202. In some cases, the actuation mechanism 208 can include a limiting element 220 which can be positioned in the handle 206 and supports the biasing element 218. Additionally or alternatively, the limiting element may act as a seal between the cannula 202 and the handle 206. For example, an arthroscopic procedure may include pressurizing the surgical cavity to expand the surgical cavity during the procedure. The limiting element 220 or separate seal (e.g., an o-ring) can isolate the recess 216 cavity from the increased pressure at the surgical site, which may help prevent movement of fluid or other material from the surgical site and into the cannula 202 or other portions of the bone wax delivery tool 200.

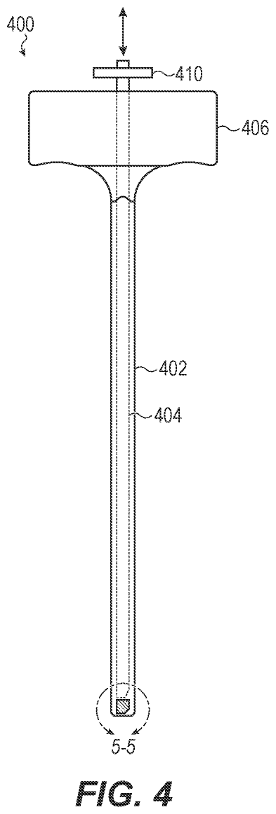

FIG. 4 shows an example bone wax delivery tool 400, which can be an example of the bone wax delivery tools described herein. The bone wax delivery tool 400 can include a cannula 402, a plunger 404, a handle 406 and an actuation mechanism (e.g., actuation mechanism 208) including an input structure 210.

In some cases, the consistency of bone wax material 401 can cause the bone wax material 401 to stick to itself and/or the bone wax delivery tool 400. This can reduce the adherence of the bone wax material to the bone, which may make it less effective at reducing bleeding from the bone and/or make the bone wax material 401 more likely to detach and float around the surgical site. The bone wax delivery tool can include features that help release the bone wax material 401 from the bone wax delivery tool 400. For example, the tip of the plunger 404 and the cannula 402 can be configured with features that help release the bone wax material 401.

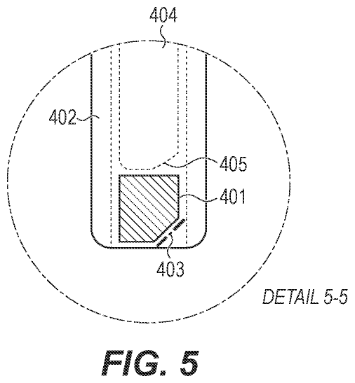

FIG. 5 shows a detailed view 5-5 of an end portion of the bone wax delivery tool 400 shown in FIG. 4. In some cases, the inside of the cannula 402 can define a first angled surface 403 and the end of the plunger 404 can have a second angled surface 405. The first angled surface 403 may extend between a sidewall of the cannula 402 and an opening in the end of the cannula 402. Accordingly, the opening of the cannula 402 can have an opening that has a reduce area as compared to embodiments that do not include the angled surface 403. The second angled surface 405 on the plunger 404 may align with the first angled surface such that the angled surface 405 of the plunger contacts the angled surface 403 of the cannula when the plunger is moved to the second position (shown in FIG. 2B). In these examples, the angled surfaces may reduce a surface area of the plunger 204 that contacts the bone wax material 401, which may reduce adhesion of the bone wax material 401 to the bone wax delivery tool 400. In other cases, the opening of the cannula and/or the end of the plunger 404 can have other configurations or shapes to help release the bone wax material 401 from the delivery tool 400.

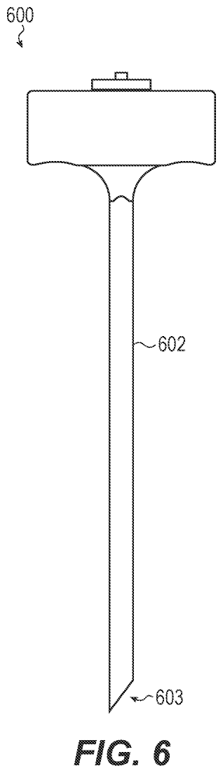

In some cases, an end of the cannula may be configured for reaching a particular location or positioning the bone wax delivery tool in a particular orientation with respect to a surgical site, which may help delivery of the bone wax material from a particular incision site and/or reduce interference with a camera or other arthroscopic tools. For example, FIG. 6 shows an example bone wax delivery tool 600, which can be an example of the bone wax delivery tools described herein. The bone wax delivery tool 600 can include a cannula 602 to has an angled end 603. The angled end 603 may allow the bone wax delivery tool 600 to be positioned at an angled orientation relative to the bone site that the bone wax material is being deposited on. In other cases, the end of the cannula can include any other suitable shape or features.

In some cases, the end of the cannula can be configured to seal around a bleeding area of the bone to deliver the bone wax material. In some cases, the tip can be angled and/or include other features that help form a seal around a bleeding portion of a bone. For example, the tip of the cannula can have a compliant material that can be compressed against the bone. In other cases, the tip of the cannula may include an edge that can be pressed into and/or cut into the bone.

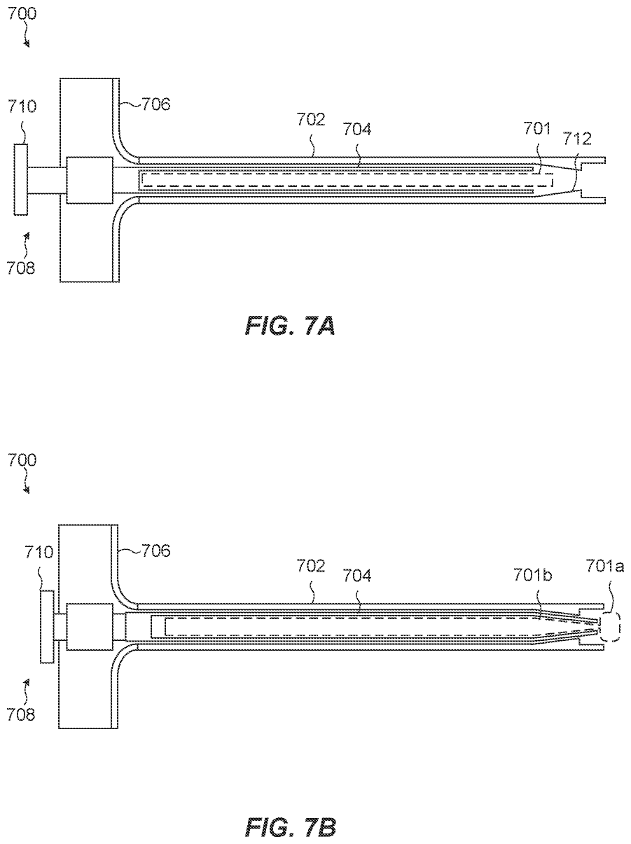

FIGS. 7A and 7B show an example bone wax delivery tool 700 that can deliver multiple doses of a bone wax material 701. The bone wax delivery tool 700 can be an example of the bone wax delivery tools described herein and include a cannula 702, a plunger 704, a handle 706 and an actuation mechanism 708 including an input structure 710.

The plunger 704 can at least partially surround the bone wax material 701. The cannula 702 can include one or more compression features 712 or the compression features 712 can be coupled to the inside of the cannula 702. Actuation of the input structure 710 by the user can cause the actuation mechanism 708 to advance the plunger 704 along the cannula 702 from a first position shown in FIG. 7A to a second position shown in FIG. 7B. As the plunger is advanced, the compression features 712 can cause the plunger 704 to compress around a portion of the bone wax material 701. This may cause a first portion of the bone wax material 701a to be separated from a second portion of the bone wax material 701b located in the cannula 702 thereby allowing the first portion of the bone wax material 701a to be expelled from the cannula 702.

Release or retraction of the input structure 710 can cause the plunger 704 to retract along the cannula 702, which may also aid release of the first portion of the bone wax material 701a. In some cases, the bone wax delivery tool can include a stop or other features that prevent the bone wax material 701 from retracting with the plunger 704. Accordingly, the release or retraction of the plunger can cause additional bone wax material 701 to be positioned from a subsequent actuation and expelled from the cannula 702. Accordingly, the bone wax delivery tool 700 may deliver multiple consecutive doses without needing to be removed from the surgical site.

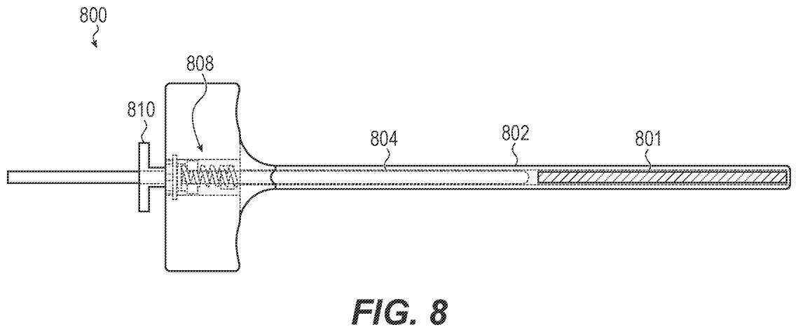

FIG. 8 shows another example of a bone wax delivery tool 800 that can deliver multiple doses of bone wax material 801. The bone wax delivery tool 800 can be an example of the bone wax delivery tools described herein and include a cannula 802, a plunger 804, a handle 806 and an actuation mechanism 808 including an input structure 810.

The bone wax material 801 can be positioned in the cannula 802 and include a bulk of material that includes multiple doses. The actuation mechanism 808 may include a ratchet mechanism that incrementally moves the plunger along the cannula 802 with each actuation stroke of the input structure 810. For example, pressing the input structure 810 toward the handle 806 can cause the plunger 804 to move toward a distal end of the cannula 802, thereby expelling a dose of bone wax material 801 from the distal end of the cannula 802. In some cases, the bone wax delivery tool 800 can include features that aid facilitate release of a dose of bone wax material, including an angled inner surface at the end of the cannula (e.g., angled surface 303 shown in FIG. 5) or an outer plunger structure and compression features (e.g., cannula 702 and compression features 712 shown in FIGS. 7A and 7B).

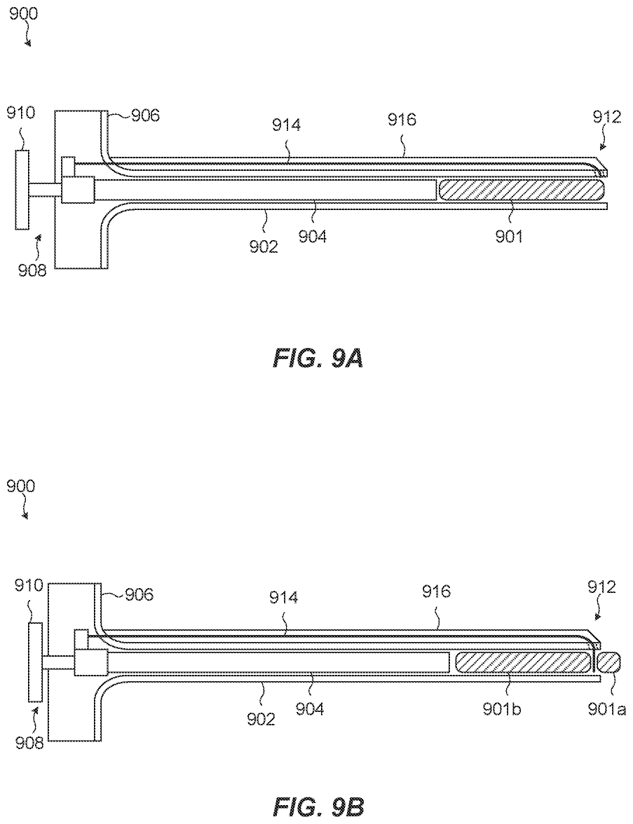

FIGS. 9A and 9B show conceptual views of an example bone wax delivery tool 900 that include a cutting mechanism 902. The bone wax delivery tool 900 can be an example of the bone wax delivery tools described herein and include a cannula 902, a plunger 904, a handle 906, and an actuation mechanism 908 including an input structure 910. The bone wax delivery tool 900 can include a cutting mechanism 912 that include a cutting element 914 and a cutting guide 916.

The cutting element 914 can be coupled to the actuation mechanism 908 and extend from the actuation mechanism to a distal portion of the cannula 902. The cutting element 914 can be contained in a guide 916 which can be coupled to the cannula 902 or formed as part of the cannula 902. The bone wax delivery tool 900 shows an example of the cutting mechanism 912 located on an outer portion of the cannula 902. In other embodiments, the cutting mechanism 912 can be located along an inner cavity of the cannula 902.

As shown in FIG. 9B, actuation of the actuation mechanism 908, for example, by pressing input structure 910 towards the handle 906, can cause both the plunger 904 to push bone wax material 901 down the cannula 902 and the cutting element 914 to move along the guide 916 to separate a first portion of bone wax material 901a from a second portion of the bone wax material 901b. The cutting mechanism 912 can be configured to control the amount of the first portion of bone wax material 901a that is separated from the second portion of bone wax material 901b and expelled from the cannula 902. Release of the input structure can cause the plunger 904 and the cutting element 914 to retract, as described herein. Accordingly, after the input structure 910 is released or otherwise retracted, the bone wax delivery tool 900 may be ready to dispense another dose of bone wax material 901.

In some cases the actuation mechanism 908 can have a ratcheting structure that advances the plunger further down the cannula 902 with each actuation event. Accordingly, as bone wax material 901 is dispensed from the cannula 902, the plunger 904 may continue to move along a length of the cannula 902 to dispense additional bone wax material 901. The cutting element 914 may be coupled to the actuation mechanism to move between a first state (retracted state) where the cutting element 914 dose not block the advance of the bone wax material 901 as shown in FIG. 9A and an second state (extended state) where the cutting element 914 extends into the cannula 902 to separate the first portion of the bone wax material 901a. Accordingly, with each actuation the cutting element 914 may move from a retracted state (shown in FIG. 9A) to an extended state (shown in FIG. 9B), and with each release the cutting element 914, may move from the extended state to the retracted state.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A bone wax delivery tool for arthroscopic procedures, comprising:
a cannula defining an inner cavity configured to hold a bone wax material;
a plunger that extends along the inner cavity of the cannula, comprising an end portion configured to contact the bone wax material, and configured to move between a first position and a second position within the cannula; and
an actuation mechanism coupled to the plunger and configured to:
in response to actuation, cause the plunger to move from the first position to the second position and expel an amount of the bone wax material from the cannula; and
in response to release of the actuation mechanism, cause the plunger to move from the second position to the first position.

2. The bone wax delivery tool of claim 1, further comprising
a handle attached to the cannula; and
an input structure coupled to an end of the plunger; wherein:
the end of the plunger extends through the handle; and
the actuation mechanism includes a key that moves within the handle and limits movement of the plunger between the first position and the second position.

3. The bone wax delivery tool of claim 1, wherein the actuation mechanism prevents the end portion of the plunger from exiting the cannula when the plunger moves to the second position.

4. The bone wax delivery tool of claim 1, wherein:
the cannula defines a tip surface at a distal end of the cannula; and
the tip surface is angled.

5. The bone wax delivery tool of claim 1, wherein:
the cannula defines a distal opening; and
the cannula defines an angled surface that extends between a portion of an inner wall of the cannula and a portion of the distal opening.

6. The bone wax delivery tool of claim 5, wherein:
the end portion of the plunger defines an angled surface; and
the angled surface of the plunger contacts the angled surface of the cannula when the plunger is actuated to the second position.

7. The bone wax delivery tool of claim 1, wherein actuation of the plunger causes the plunger to rotate with respect to the cannula.

8. The bone wax delivery tool of claim 1, wherein, the actuation mechanism comprises a spring that biases the plunger to the first position.

9. A bone wax delivery tool for arthroscopic procedures, comprising:
a cannula defining an inner cavity configured to hold a bone wax material;
a plunger positioned in the inner cavity of the cannula; and
an actuation mechanism coupled to the plunger and configured to:
in response to a first actuation, cause the plunger to advance along the cannula and expel a first portion of the bone wax material from the cannula; and
in response to a second actuation, cause the plunger to advance along the cannula and expel a second portion of bone wax material from the cannula.

10. The bone wax delivery tool of claim 9, wherein:
the bone wax material extends along the inner cavity of the cannula;
the plunger is positioned at least partially around the bone wax material;
each actuation moves the plunger from a first position and to a second position; and
each release of the actuation mechanism returns the plunger to the first position.

11. The bone wax delivery tool of claim 10, wherein movement of the plunger to the second position causes the plunger to separate an expelled portion of the bone wax material from a rest of the bone wax material.

12. The bone wax delivery tool of claim 9, wherein:
the bone wax material extends along the inner cavity of the cannula;
the plunger comprise an end portion configured to contact the bone wax material; and
each actuation advances the plunger along the cannula.

13. The bone wax delivery tool of claim 9, further comprising:
a handle attached to the cannula; and
an input structure coupled to the cannula and positioned outside the handle; wherein:
each actuation of the input structure causes the input structure to move from a first position to a second position; and
each release of the actuation component causes the input structure to return to the first position.

14. The bone wax delivery tool of claim 9, wherein:
the cannula defines a distal opening; and
the cannula defines an angled surface that extends between a portion of an inner wall of the cannula and a portion of the distal opening.

15. The bone wax delivery tool of claim 9, wherein:
the cannula defines a tip surface at a distal end of the cannula; and
the tip surface is angled.

16. A bone wax delivery tool for arthroscopic procedures, comprising:
a cannula defining an inner cavity configured to hold a bone wax material;
a plunger that extends along the inner cavity of the cannula and configured to contact the bone wax material;
a handle attached to the cannula;
an actuation mechanism coupled to the plunger and comprising an input structure positioned outside the handle and configured to move between a first position and a second position, the actuation mechanism configured to:
in response a press operation to the input structure, the input structure moves from the first position to the second position and causes the plunger to expel a defined amount of the bone wax material from the cannula; and
in response to release of the input structure, the input structure returns to the first position.

17. The bone wax delivery tool of claim 16, wherein:
the handle is positioned at an end of cannula;
the plunger extends through the handle; and
the input structure is attached to an end of the plunger.

18. The bone wax delivery tool of claim 16, further comprising a cutting mechanism coupled to the actuation mechanism, wherein in response to the press operation to the input structure, the actuation mechanism causes the defined amount of the bone wax material to be separated from the bone wax material in the cannula.

19. The bone wax delivery tool of claim 16, wherein:
the cutting mechanism comprises a cutting element that extends from the actuation mechanism and to the distal end of the cannula.

20. The bone wax delivery tool of claim 16, further comprising a seal that engages with the plunger and is configured to prevent movement of fluid from surgery site and through the cannula.
